SEQUENCE LISTING (10 sequences)

SEQ ID NO: 1 - DNA, 20 bp, synthetic construct
  source 1..20: mol_type = other DNA
aatgatacggcgaccaccga

SEQ ID NO: 2 - DNA, 29 bp, synthetic construct
  source 1..29: mol_type = other DNA
aatgatacggcgaccaccgagatctacac

SEQ ID NO: 3 - DNA, 20 bp, synthetic construct
  source 1..20: mol_type = other DNA
caagcagaagacggcatacg

SEQ ID NO: 4 - DNA, 24 bp, synthetic construct
  source 1..24: mol_type = other DNA
caagcagaagacggcatacgagat

SEQ ID NO: 5 - DNA, 30 bp, synthetic construct
  source 1..30: mol_type = other DNA
  modified_base 1: mod_base = OTHER; note = Phosphorothioate
ttttttttttaatgatacggcgaccaccga

SEQ ID NO: 6 - DNA, 31 bp, synthetic construct
  source 1..31: mol_type = other DNA
  modified_base 1: mod_base = OTHER; note = Phosphorothioate
ttttttttttcaagcagaagacggcatacga

SEQ ID NO: 7 - DNA, 33 bp, synthetic construct
  source 1..33: mol_type = other DNA
acactctttccctacacgacgctcttccgatct

SEQ ID NO: 8 - DNA, 20 bp, synthetic construct
  source 1..20: mol_type = other DNA
acactctttccctacacgac

SEQ ID NO: 9 - DNA, 34 bp, synthetic construct
  source 1..34: mol_type = other DNA
gtgactggagttcagacgtgtgctcttccgatct

SEQ ID NO: 10 - DNA, 21 bp, synthetic construct
  source 1..21: mol_type = other DNA
gtgactggagttcagacgtgt

(19) United States
(12) Patent Application Publication
(10) Pub. No.: US 2025/0368985 A1
(43) Pub. Date: Dec. 4, 2025

(54) MATERIALS AND METHODS FOR PREPARATION OF A SPATIAL TRANSCRIPTOMICS LIBRARY

(71) Applicant: ILLUMINA, INC., San Diego, CA (US)

(72) Inventors: Yue Ding, Mountain View, CA (US); Lena Storms, San Diego, CA (US); Craig April, San Diego, CA (US); Yao Xiao, San Diego, CA (US); Anustup Poddar, San Diego, CA (US); Brittany Flowers, San Diego, CA (US); Mats Ekstrand, San Diego, CA (US)

(21) Appl. No.: 18/875,223

(22) PCT Filed: Dec. 29, 2023
(86) PCT No.: PCT/US23/86361

§ 371 Date: Dec. 16, 2024

Related U.S. Application Data
(60) Provisional application No. 63/477,730, filed on Dec. 29, 2022.

(51) Int. Cl. C12N 15/10

(52) U.S. Cl. CPC C12N 15/1093

(57) ABSTRACT

The present disclosure relates, in general, to materials and methods for improving RNA capture in situ from tissue samples and improved methods for synthesizing cDNA from the captured RNA.

### CROSS REFERENCE TO RELATED APPLICATIONS

The present application claims the priority benefit of U.S. Provisional Patent Application No. 63/477,730, filed Dec. 29, 2022, incorporated by reference herein in its entirety.

### INCORPORATION BY REFERENCE OF SEQUENCE DISCLOSURE

The Sequence Listing, which is a part of the present disclosure, is submitted concurrently with the specification as a computer readable file. The name of the file containing the Sequence Listing is “IP-2526_SeqListing.xml”, which was created on Dec. 21, 2023, and is 10,966 bytes in size. The subject matter of the Sequence Listing is incorporated herein in its entirety by reference.

### FIELD OF THE DISCLOSURE

The present disclosure relates, in general, to improved methods for preparing RNA from a tissue sample and preparation of a spatial transcriptomics library from the isolated RNA.

### BACKGROUND OF THE DISCLOSURE

Spatial transcriptomics enables highly multiplexed, spatially localized gene expression analysis from fresh frozen and formalin-fixed paraffin-embedded (FFPE) tissue samples. However, due to the freezing process or the fixation process of FFPE tissue, fragmentation, degradation, and crosslinking can alter the quality and quantity of RNA and DNA for transcriptomics library preparation. Current on-market spatial workflows capture and convert<1% mRNA within a tissue section.

### SUMMARY OF THE DISCLOSURE

Presented here are methods to generate higher capture and spatial library conversion from preserved tissue samples, e.g., frozen or FFPE tissue samples. In situ polyadenylation can enable capture of fragmented FFPE RNA on oligo-dT surface. Also provided herein are improved methods to synthesize cDNA from isolated mRNA transcripts to improve the overall synthesis and alignment quality of the mRNA sequences and preparation of a spatial transcriptomics library.

The present disclosure provides a method for isolating RNA from a sample comprising (a) contacting total RNA isolated from the sample with polynucleotide kinase (PNK) to modify 3′ phosphate to a hydroxyl group to generate end repaired total RNA; (b) contacting the end repaired total RNA with polyadenylate polymerase (PAP) and adenosine nucleotides to generate polyadenylated total RNA; (c) capturing the polyadenylated total RNA on a substrate comprising one or more oligonucleotides comprising poly T sequences; and (d) eluting the polyadenylated total RNA from the substrate.

In various embodiments, the method further comprises quantifying the total RNA. In some aspects, RNA is quantified using Qubit or RT-qPCR.

Also provided is a method for preparing an RNA library from a tissue sample comprising, (a) contacting total RNA isolated from the sample with polynucleotide kinase (PNK) to modify 3′ phosphate to a hydroxyl group to generate end repaired total RNA; (b) contacting the end repaired total RNA with polyadenylate polymerase (PAP) and adenosine nucleotides to generate polyadenylated total RNA; (c) releasing the polyadenylated total RNA from the tissue sample; (d) capturing the polyadenylated total RNA on a substrate comprising one or more oligonucleotides comprising a poly T sequence; (e) generating an RNA library from the polyadenylated total RNA using a RNA library prep kit.

In various embodiments, the releasing is done by lysing and/or permeabilizing the tissue sample.

In various embodiments, the RNA comprises rRNA and/or mRNA.

Further contemplated is a method for preparing an mRNA transcriptome library from a tissue sample comprising, (a) contacting total RNA isolated from the sample with polynucleotide kinase (PNK) to modify 3′ phosphate to a hydroxyl group to generate end repaired total RNA; (b) contacting the end repaired total RNA with polyadenylate polymerase (PAP) and adenosine nucleotides to generate polyadenylated total RNA; (c) releasing the polyadenylated total RNA from the tissue sample; (d) capturing the polyadenylated total RNA on a substrate comprising one or more oligonucleotides comprising a poly T sequence; (e) depleting ribosomal RNA from the total RNA to leave polyadenylated mRNA; (f) generating an mRNA library from the polyadenylated mRNA using a mRNA library prep kit.

In various embodiments, the substrate is a bead, a bead array, a spotted array, a flow cell (e.g., a clustered flow cell), clustered particles arranged on a surface of a chip, a film, and a plate (e.g., a multi-well plate).

In various embodiments, the sample is a fresh frozen tissue sample or a formalin-fixed paraffin embedded (FFPE) sample.

In various embodiments, releasing comprises contacting the sample with a lysis buffer, a pemeabilization buffer and/or a reagent to deparaffinize a FFPE sample. For example, when the sample is a FFPE sample on a slide, the method may comprise permeabilization and collagenase treatment of the sample on the slide prior to contacting the RNA with PNK. The method may further comprise decrosslinking the FFPE sample, optionally wherein the decrosslinking is carried out using TE buffer, pH 9.

In various embodiments, after polyadenylation, the polyA tail is between 3 and 50 nucleotides.

In various embodiments, generating the RNA library comprises the steps of eluting the polyadenylated total RNA from the substrate and generating the RNA library from the eluted polyadenylated RNA library using a RNA library prep kit. In some embodiments, generating the RNA library comprises, i) contacting the isolated RNA with a reverse transcriptase (RT) or DNA polymerase to generate a first cDNA strand complementary to the RNA; ii) contacting the first cDNA strand with a reverse transcriptase (RT) or DNA polymerase to generate a second cDNA strand complementary to the first cDNA strand; iii) amplifying the second strand cDNA to form a PCR template and isolating the PCR template; and iv) generating an RNA library from the PCR templates.

In various embodiments, one or more of a first clustering sequence, an index sequence, and/or a Read 2 sequence are added during or prior to second strand synthesis.

In various embodiments, the RNA library is an mRNA library.

In various embodiments, the PCR templates are further processed by tagmentation to generate a spatial transcriptomics library. In some embodiments, the tagmentation comprises on bead tagmentation, wherein the bead comprises a plurality of bead-linked transposomes (BLT). In some embodiments, the BLT comprises i) a plurality of oligonucleotides comprising a first clustering sequence (P7), a first index sequence and a Read 1 sequencing primer (Rd1 SP) and ii) a plurality of oligonucleotides comprising a second clustering sequence (P5), a second index sequence and a Read 2 sequencing primer (Rd2 SP).

Also provided by the disclosure is a method for improving capture efficiency of mRNA transcripts for in situ mRNA transcript library preparation comprising, (a) capturing mRNA transcripts from a sample on a substrate (b) contacting the substrate with a high processivity reverse transcriptase (RT) to generate a first cDNA strand complementary to the mRNA transcripts; (c) contacting the first cDNA strand with a DNA polymerase to generate a second cDNA strand complementary to the first cDNA strand; (d) amplifying the second strand cDNA to form a PCR template and isolating the PCR template.

In other embodiments, the method for improving capture efficiency of mRNA transcripts for in situ mRNA transcript library preparation comprises, (a) capturing mRNA transcripts from a sample on a substrate; (b) contacting the substrate with a high processivity reverse transcriptase (RT) to generate a first cDNA strand complementary to the mRNA transcripts; (c) contacting the first cDNA strand with the high processivity reverse transcriptase (RT) or high processivity DNA polymerase to generate a second cDNA strand complementary to the first cDNA strand; and (d) amplifying the second strand cDNA to form a PCR template and isolating the PCR template.

In still other embodiments, provided is a method for improving the nucleotide length of polynucleotides used in generating an in situ transcriptome library comprising, (a) capturing mRNA transcripts from a sample on a substrate; (b) contacting the substrate with a high processivity reverse transcriptase (RT) to generate a first cDNA strand complementary to the mRNA transcripts; (c) contacting the first cDNA strand with a high processivity reverse transcriptase (RT) or high processivity DNA polymerase to generate a second cDNA strand complementary to the first cDNA strand; and (d) amplifying the second strand cDNA to form a PCR template and isolating the PCR template.

In various embodiments, the high processivity RT is Superscript IV, thermostable group II intron RT (TGIRT), or marathon RT. In various embodiments, the high processivity DNA polymerase is Klenow exo-, Bst 3.0, or phi29. In various embodiments, the DNA polymerase lacks both 5→3′ and 3′→5 exonuclease activity.

Also provided is a method for preparing an mRNA transcriptome library from a tissue sample comprising, (a) contacting total RNA isolated from the sample with polynucleotide kinase (PNK) to modify 3′ phosphate to a hydroxyl group to generate end repaired total RNA; (b) contacting the total RNA with polynucleotide kinase (PNK) to modify a 3′ phosphate to a hydroxyl group to generate end repaired total RNA; (c) contacting the end repaired total RNA with polyadenylate polymerase (PAP) and adenosine nucleotides to generate polyadenylated total RNA; (d) releasing the polyadenylated total RNA from the tissue sample; (e) capturing the polyadenylated total RNA on a substrate comprising one or more oligonucleotides comprising a poly T sequence; (f) depleting ribosomal RNA from the total RNA leaving polyadenylated mRNA; (g) contacting the polyadenylated mRNA with a high processivity reverse transcriptase (RT) to generate a first cDNA strand complementary to the mRNA transcripts; (h) contacting the first cDNA strand with a high processivity reverse transcriptase (RT) or high processivity DNA polymerase to generate a second cDNA strand complementary to the first cDNA strand to generate PCR templates; (i) eluting the PCR templates; and (j) generating an mRNA library from the PCR templates.

In various embodiments, the sample is a fresh frozen tissue sample or a formalin-fixed paraffin embedded (FFPE) sample. In various embodiments, when the sample is a FFPE sample on a slide, the method may comprise permeabilization and collagenase treatment of the sample on the slide prior to contacting the RNA with PNK. Optionally, the method further comprises decrosslinking the FFPE sample, optionally wherein the decrosslinking is carried out using TE buffer, pH 9.

In various embodiments, generating the RNA library comprises, i) contacting the isolated RNA with a reverse transcriptase (RT) to generate a first cDNA strand complementary to the RNA; ii) contacting the first cDNA strand with a reverse transcriptase (RT) or DNA polymerase to generate a second cDNA strand complementary to the first cDNA strand; iii) amplifying the second strand cDNA to form a PCR template and isolating the PCR template; iv) generating an RNA library from the PCR templates.

In various embodiments, one or more of a first clustering sequence, an index sequence, and/or a Read 1 or Read 2 sequence are added during or prior to second strand synthesis.

In various embodiments, the RNA library is an mRNA library.

In various embodiments, the PCR templates are further processed by tagmentation to generate a spatial transcriptomics library. In some embodiments, the tagmentation comprises on bead tagmentation, wherein the bead comprises a plurality of bead-linked transposomes (BLT). In various embodiments, the BLT comprises, i) a plurality of oligonucleotides comprising a first clustering sequence (P7), a first index sequence and a Read 1 sequencing primer (Rd1 SP); and ii) a plurality of oligonucleotides comprising a second clustering sequence (P5), a second index sequence and a Read 2 sequencing primer (Rd2 SP).

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “various embodiments”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination.

Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1. Schematic illustrating polyadenylation in formalin-fixed paraffin embedded (FFPE) and fresh frozen (FF) tissue.

FIG. 2. Workflow schematic to test efficiency of polyadenylation on total extracted RNA on FFPE and fresh frozen tissue.

FIG. 3. Protocol for polyadenylation study on extracted RNA.

FIG. 4. % capture of RNA on oligo-dT beads from polyadenylation in-tube workflow.

FIG. 5. Captured RNA run on high sensitivity RNA screentape.

FIG. 6. Workflow schematic to test efficiency of in situ polyadenylation on FFPE and fresh frozen tissue.

FIG. 7. Detailed protocol for polyadenylation study on FFPE and fresh frozen tissue.

FIG. 8. Probe-based RT-PCR of captured RNA from in situ polyadenylation experiment.

FIG. 9. Captured RNA yields quantified with high sensitivity RNA Qubit kit.

FIG. 10. Schematic of library preparation and sequencing libraries.

FIG. 11. Results of PolyA trimming of sequenced libraries.

FIG. 12A-12C. FF and FFPE sequencing data from basespace RNA-seq alignment app. FIG. 12A.) Polyadenylation shifts 3′ bias transcript coverage. FIG. 12B.) Polyadenylation increases insert size. FIG. 12C.) Polyadenylation increases % reads aligning to coding region for FFPE.

FIG. 13. Analysis of cDNA size after use of SSIV in 2^{nd} strand synthesis

FIG. 14. Improved cDNA length when using SSIV as the polymerase for 2nd strand synthesis.

FIG. 15. cDNA preparation using SSIV for 1st strand and 2nd strand synthesis.

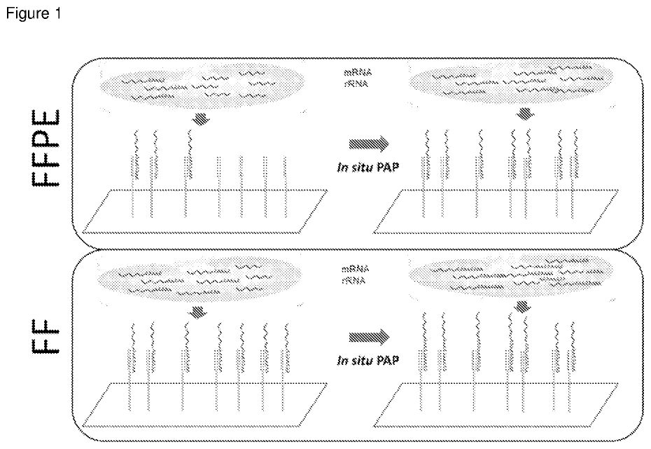

### DETAILED DESCRIPTION

Isolating mRNA from preserved tissue samples and converting mRNA to cDNA on a flat surface presents a number of problems, including lower quality mRNA transcripts isolated from the tissue samples, shorter synthesized cDNA fragments (<450 bp) in library preparation products and a high percentage of polyA presence in cDNA regions in the final sequencing products. These issues result in a subsequent low mapping rate to exonic mRNA transcript regions in RNA-seq alignment.

To solve this problem, it was hypothesized that an improved method to generate higher capture and spatial library conversion from FFPE tissue samples was needed. In situ polyadenylation can enable capture of fragmented FFPE RNA on oligo-dT surface. Also needed was an improvement in synthesizing the cDNA using reverse transcriptase (RTase) with faster processivity and thermostability, e.g., Superscript IV, to 1) replace the well-established DNA polymerase (Klenow exo−) used in second strand synthesis step that is usually done with DNA polymerases, and optionally combine with 2) replacing the slower RTase used in first strand synthesis (e.g., maxima H−) with a high processivity RT, to achieve longer cDNA length in shorter workflow time frame

### Definitions

Unless otherwise stated, the following terms used in this application, including the specification and claims, have the definitions given below.

As used in this specification and the appended claims, the singular forms “a”, “an” and “the” include plural referents unless the content clearly dictates otherwise. Thus, for example, reference to “a capture probe” includes a mixture of two or more capture probes, and the like.

The term “about,” particularly in reference to a given quantity, is meant to encompass deviations of plus or minus five percent.

As used herein, the terms “includes,” “including,” “includes,” “including,” “contains,” “containing,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter.

As used herein, the terms “address,” “tag,” or “index,” when used in reference to a nucleotide sequence is intended to mean a unique nucleotide sequence that is distinguishable from other indices as well as from other nucleotide sequences within polynucleotides contained within a sample. A nucleotide “address,” “tag,” or “index” can be a random or a specifically designed nucleotide sequence. An “address,” “tag,” or “index” can be of any desired sequence length so long as it is of sufficient length to be unique nucleotide sequence within a plurality of indices in a population and/or within a plurality of polynucleotides that are being analyzed or interrogated. A nucleotide “address,” “tag,” or “index” of the disclosure is useful, for example, to be attached to a target polynucleotide to tag or mark a particular species for identifying all members of the tagged species within a population. Accordingly, an index is useful as a barcode where different members of the same molecular species can contain the same index and where different species within a population of different polynucleotides can have different indices.

A tag/index/barcode sequence can be unique to a single nucleic acid species in a population or can be shared by several different nucleic acid species in a population. For example, each nucleic acid probe in a population can include different tag/index/barcode sequences from all other nucleic acid probes in the population. Alternatively, each nucleic acid probe in a population can include different tag/index/barcode sequences from some or most other nucleic acid probes in a population. For example, each probe in a population can have a tag/index/barcode that is present for several different probes in the population even though the probes with the common tag/index/barcode differ from each other at other sequence regions along their length. In particular embodiments, one or more tag/index/barcode sequences that are used with a biological specimen are not present in the genome, transcriptome or other nucleic acids of the biological specimen. For example, tag/index/barcode sequences can have less than 80%, 70%, 60%, 50% or 40% sequence identity to the nucleic acid sequences in a particular biological specimen.

As used herein, a “spatial address,” “spatial tag”, “spatial barcode”, “spatial barcode sequence” or “spatial index,” when used in reference to a nucleotide sequence, means an address, tag, barcode or index encoding spatial information related to the region or location of origin of an addressed, tagged, barcoded or indexed nucleic acid in a tissue sample. The sequence can be a naturally occurring sequence or a sequence that does not occur naturally in the organism from which the barcoded nucleic acid was obtained.

As used herein, the term “substrate” is intended to mean a solid support or support structure. The term includes any material that can serve as a solid or semi-solid foundation for creation of features such as wells for the deposition of biopolymers, including nucleic acids, polypeptide and/or other polymers. Non-limiting examples of substrates include a bead array, a spotted array, clustered particles arranged on a surface of a chip, a film, a multi-well plate, and a flow cell. A substrate as provided herein is modified, for example, or can be modified to accommodate attachment of biopolymers by a variety of methods well known to those skilled in the art. Exemplary types of substrate materials include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, a variety of polymers other than those exemplified above and multiwell microtiter plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon.

Those skilled in the art will know or understand that the composition and geometry of a substrate as provided herein can vary depending on the intended use and preferences of the user. Therefore, although planar substrates such as slides, chips wafers or beads are useful for microarrays, those skilled in the art will understand that a wide variety of other substrates exemplified herein or well known in the art also can be used in the methods and/or compositions herein.

In some embodiments, the solid support comprises one or more surfaces that are accessible to contact with reagents, beads, or analytes. The surface can be substantially flat or planar. Alternatively, the surface can be rounded or contoured. Example contours that can be included on a surface are wells (e.g., microwells or nanowells), depressions, pillars, ridges, channels or the like. Example materials that can be used as a surface include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon, carbon-fibre; metal; inorganic glass; optical fibre bundle, or a variety of other polymers. A single material or mixture of several different materials can form a surface useful in certain examples. In some examples, a surface comprises wells (e.g., microwells or nanowells). In some aspects, the surface comprises wells in an array of wells (e.g., microwells or nanowells) on glass, silicon, plastic or other suitable solid supports with patterned, covalently-linked gel such as poly(N-(5-azidoacetamidylpentyl) acrylamide-coacrylamide) (PAZAM, see, for example, U.S. Pat. App. Pub. No. 2014/0079923 A1, which is incorporated herein by reference). In some examples, a support structure can include one or more layers.

In some embodiments, the solid support comprises one or more surfaces of a flowcell. The term “flowcell” as used herein refers to a chamber comprising a solid surface across which one or more fluid reagents can be flowed. The flow cell can be an ordered or random flow cell. Examples of flowcells and related fluidic systems and detection platforms that can be readily used in the methods of the present disclosure are described, for example, in Bentley et al., Nature 456:53-59 (2008), WO04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, the solid support includes a patterned surface. A “patterned surface” refers to an arrangement of different regions in or on an exposed layer of a solid support. For example, one or more of the regions can be features where one or more amplification primers are present. The features can be separated by interstitial regions where amplification primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Ser. No. 13/661,524 or US Pat. App. Publ. No. 2012/0316086, or International Patent Publication WO 2017/019456, each of which is incorporated herein by reference.

As used herein, the term “immobilized” when used in reference to a nucleic acid is intended to mean direct or indirect attachment to a solid support via covalent or non-covalent bond(s). In certain embodiments, covalent attachment can be used, but all that is required is that the nucleic acids remain stationary or attached to a support under conditions in which it is intended to use the support, for example, in applications requiring nucleic acid amplification and/or sequencing. Oligonucleotides to be used as capture primers or amplification primers can be immobilized such that a 3′-end is available for enzymatic extension and at least a portion of the sequence is capable of hybridizing to a complementary sequence.

Immobilization can occur via hybridization to a surface attached oligonucleotide, in which case the immobilized oligonucleotide or polynucleotide can be in the 3′-5′ orientation. Alternatively, immobilization can occur by means other than base-pairing hybridization, such as the covalent attachment set forth above

Exemplary covalent linkages include, for example, those that result from the use of click chemistry techniques. Exemplary non-covalent linkages include, but are not limited to, non-specific interactions (e.g., hydrogen bonding, ionic bonding, van der Waals interactions etc.) or specific interactions (e.g., affinity interactions, receptor-ligand interactions, antibody-epitope interactions, avidin-biotin interactions, streptavidin-biotin interactions, lectin-carbohydrate interactions, etc.). Exemplary linkages are set forth in U.S. Pat. Nos. 6,737,236; 7,259,258; 7,375,234 and 7,427,678; and US Pat. Pub. No. 2011/0059865 A1, each of which is incorporated herein by reference.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “plurality” is intended to mean a population of two or more different members. Pluralities can range in size from small, medium, large, to very large. The size of small plurality can range, for example, from a few members to tens of members. Medium sized pluralities can range, for example, from tens of members to about 100 members or hundreds of members. Large pluralities can range, for example, from about hundreds of members to about 1000 members, to thousands of members and up to tens of thousands of members. Very large pluralities can range, for example, from tens of thousands of members to about hundreds of thousands, a million, millions, tens of millions and up to or greater than hundreds of millions of members. Therefore, a plurality can range in size from two to well over one hundred million members as well as all sizes, as measured by the number of members, in between and greater than the above exemplary ranges. An exemplary number of features within a microarray includes a plurality of about 500,000 or more discrete features within 1.28 cm². Exemplary nucleic acid pluralities include, for example, populations of about 1×10⁵, 5×10⁵ and 1×10⁶ or more different nucleic acid species. Accordingly, the definition of the term is intended to include all integer values greater than two. An upper limit of a plurality can be set, for example, by the theoretical diversity of nucleotide sequences in a nucleic acid sample.

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid are known in the art. The term “target,” when used in reference to a nucleic acid, is intended as a semantic identifier for the nucleic acid in the context of a method or composition set forth herein and does not necessarily limit the structure or function of the nucleic acid beyond what is otherwise explicitly indicated. Particular forms of nucleic acids may include all types of nucleic acids found in an organism as well as synthetic nucleic acids such as polynucleotides produced by chemical synthesis.

Particular examples of nucleic acids that are applicable for analysis through incorporation into microarrays produced by methods as provided herein include genomic DNA (gDNA), expressed sequence tags (ESTs), DNA copied messenger RNA (cDNA), RNA copied messenger RNA (cRNA), mitochondrial DNA or genome, RNA, messenger RNA (mRNA), ribosomal RNA (rRNA) and/or other populations of RNA. Fragments and/or portions of these exemplary nucleic acids also are included within the meaning of the term as it is used herein.

As used herein, the term “double-stranded,” when used in reference to a nucleic acid molecule, means that substantially all of the nucleotides in the nucleic acid molecule are hydrogen bonded to a complementary nucleotide. A partially double stranded nucleic acid can have at least 10%, 25%, 50%, 60%, 70%, 80%, 90% or 95% of its nucleotides hydrogen bonded to a complementary nucleotide.

As used herein, the term “single-stranded,” when used in reference to a nucleic acid molecule, means that essentially none of the nucleotides in the nucleic acid molecule are hydrogen bonded to a complementary nucleotide.

As used herein, the term “capture primers” or “capture probe” is intended to mean an oligonucleotide having a nucleotide sequence that is capable of specifically annealing to a single stranded polynucleotide sequence to be analyzed or subjected to a nucleic acid interrogation under conditions encountered in a primer annealing step of, for example, an amplification or sequencing reaction. The terms “nucleic acid,” “polynucleotide” and “oligonucleotide” are used interchangeably herein. The different terms are not intended to denote any particular difference in size, sequence, or other property unless specifically indicated otherwise. For clarity of description the terms can be used to distinguish one species of nucleic acid from another when describing a particular method or composition that includes several nucleic acid species.

As used herein, the term “gene-specific” or “target specific” when used in reference to a capture probe or other nucleic acid is intended to mean a capture probe or other nucleic acid that includes a nucleotide sequence specific to a targeted nucleic acid, e.g., a nucleic acid from a tissue sample, namely a sequence of nucleotides capable of selectively annealing to an identifying region of a targeted nucleic acid. Gene-specific capture probes can have a single species of oligonucleotide, or can include two or more species with different sequences. Thus, the gene-specific capture probes can be two or more sequences, including 3, 4, 5, 6, 7, 8, 9 or 10 or more different sequences. The gene-specific capture probes can comprise a gene-specific capture primer sequence and a universal capture probe sequence. Other sequences such as sequencing primer sequences and the like also can be included in a gene-specific capture primer.

As used herein “unique molecular index”, “unique molecular identifier” or “UMI”, when used in reference to a capture probe or other nucleic acid is intended to refer to a portion of a probe useful as a molecular barcode to uniquely tag each molecule in a sample library. A UMI may be denoted as “NNNN . . . ” in a string of nucleic acids to designate that portion of the oligonucleotide as the UMI. A UMI may be from 6 to 20 nucleotides or more in length. In some aspects, the UMI comprises a spatial barcode.

In comparison, the term “universal” when used in reference to a capture probe or other nucleic acid is intended to mean a capture probe or nucleic acid having a common nucleotide sequence among a plurality of capture probes. A common sequence can be, for example, a sequence complementary to the same adapter sequence. Universal capture probes are applicable for interrogating a plurality of different polynucleotides without necessarily distinguishing the different species whereas gene-specific capture primers are applicable for distinguishing the different species.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. An amplicon can be a nucleic acid molecule having a single copy of a particular nucleotide sequence (e.g., a PCR product) or multiple copies of the nucleotide sequence (e.g., a concatameric product of RCA). A first amplicon of a target nucleic acid can be a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

The number of template copies or amplicons that can be produced can be modulated by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. The number of copies of a nucleic acid template can be at least 1, 10, 100, 200, 500, 1000, 2000, 3000, 4000, 5000, 6000, 7000, 8000, 9000 and 10,000 copies, and can be varied depending on the particular application.

“Processivity” refers to the ability of reverse transcriptase or DNA polymerase to carry out DNA synthesis on a template DNA without frequent dissociation or release of the template strand, and can be measured by the average number of nucleotides added by an enzyme. See e.g., Zhuang et al., Biochim Biophys Acta. 2010 May; 1804 (5): 1081-1093. In some embodiments, a high processivity enzyme can process tens to hundreds of bases per second.

As used herein, the term “complementary” when used in reference to a polynucleotide is intended to mean a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application, and can be routinely determined by persons skilled in the art, without undue experimentation.

As used herein, the term “hybridization” refers to the process in which two single-stranded polynucleotides bind non-covalently to form a stable double-stranded polynucleotide. A resulting double-stranded polynucleotide is a “hybrid” or “duplex.” Hybridization conditions will typically include salt concentrations of less than about 1 M, more usually less than about 500 mM and may be less than about 200 mM. A hybridization buffer includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are usually performed under stringent conditions, i.e., conditions under which a probe will hybridize to its target subsequence but will not hybridize to the other, uncomplimentary sequences. Stringent conditions are sequence-dependent and are different in different circumstances, and may be determined routinely by those skilled in the art.

As used herein, the term “dNTP” refers to deoxynucleoside triphosphates. NTP refers to ribonucleotide triphosphates. The purine bases (Pu) include adenine (A), guanine (G) and derivatives and analogs thereof. The pyrimidine bases (Py) include cytosine (C), thymine (T), uracil (U) and derivatives and analogs thereof. Examples of such derivatives or analogs, by way of illustration and not limitation, are those which are modified with a reporter group, biotinylated, amine modified, radiolabeled, alkylated, and the like and also include phosphorothioate, phosphite, ring atom modified derivatives, and the like. The reporter group can be a fluorescent group such as fluorescein, a chemiluminescent group such as luminol, a terbium chelator such as N-(hydroxyethyl)ethylenediaminetriacetic acid that is capable of detection by delayed fluorescence, and the like.

As used herein, the terms “ligation,” “ligating,” and grammatical equivalents thereof are intended to mean to form a covalent bond or linkage between the termini of two or more nucleic acids, e.g., oligonucleotides and/or polynucleotides, typically in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide. Template driven ligation reactions are described in the following references: U.S. Pat. Nos. 4,883,750; 5,476,930; 5,593,826; and 5,871,921, incorporated herein by reference in their entireties. The term “ligation” also encompasses non-enzymatic formation of phosphodiester bonds, as well as the formation of non-phosphodiester covalent bonds between the ends of oligonucleotides, such as phosphorothioate bonds, disulfide bonds, and the like.

As used herein, the term “each,” when used in reference to a collection of items, is intended to identify an individual item in the collection but does not necessarily refer to every item in the collection unless the context clearly dictates otherwise.

As used herein, the term “extend,” when used in reference to a nucleic acid, is intended to mean addition of at least one nucleotide or oligonucleotide to the nucleic acid. In particular embodiments one or more nucleotides can be added to the 3′ end of a nucleic acid, for example, via polymerase catalysis (e.g., DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g., ligase catalysis) methods. A nucleic acid can be extended in a template directed manner, whereby the product of extension is complementary to a template nucleic acid that is hybridized to the nucleic acid that is extended.

Provided herein are arrays for and methods of spatial detection and analysis (e.g., mutational analysis or single nucleotide variation (SNV) detection as well as indel detection) of nucleic acid in a tissue sample. The arrays described herein can comprise a substrate on which a plurality of capture probes are immobilized such that each capture probe occupies a distinct position on the array. Some or all of the plurality of capture probes can comprise a unique positional tag (i.e., a spatial address or indexing sequence). A spatial address can describe the position of the capture probe on the array. The position of the capture probe on the array can be correlated with a position in the tissue sample.

As used herein, the term “poly T” or “poly A,” when used in reference to a nucleic acid sequence, is intended to mean a series of two or more thiamine (T) or adenine (A) bases, respectively. A poly T or poly A can include at least about 2, 5, 8, 10, 12, 15, 18, 20 or more of the T or A bases, respectively. Alternatively or additionally, a poly T or poly A can include at most about, 30, 20, 18, 15, 12, 10, 8, 5 or 2 of the T or A bases, respectively.

As used herein, the term “tagmentation,” “tagment,” or “tagmenting” refers to transforming a nucleic acid, e.g., a DNA, into adaptor-modified templates in solution ready for cluster formation and sequencing by the use of transposase mediated fragmentation and tagging. This process often involves the modification of the nucleic acid by a transposome complex comprising transposase enzyme complexed with adaptors comprising transposon end sequence. Tagmentation results in the simultaneous fragmentation of the nucleic acid and ligation of the adaptors to the 5′ ends of both strands of duplex fragments. Following a purification step to remove the transposase enzyme, additional sequences are added to the ends of the adapted fragments by PCR.

A “transposase” means an enzyme that is capable of forming a functional complex with a transposon end-containing composition (e.g., transposons, transposon ends, transposon end compositions) and catalyzing insertion or transposition of the transposon end-containing composition into the double-stranded target nucleic acid with which it is incubated, for example, in an in vitro transposition reaction. A transposase as presented herein can also include integrases from retrotransposons and retroviruses. Transposases, transposomes and transposome complexes are generally known to those of skill in the art, as exemplified by the disclosure of US Pat. Publ. No. 2010/0120098, the content of which is incorporated herein by reference in its entirety. Although many embodiments described herein refer to Tn5 transposase and/or hyperactive Tn5 transposase, it will be appreciated that any transposition system that is capable of inserting a transposon end with sufficient efficiency to 5′-tag and fragment a target nucleic acid for its intended purpose can be used in the present invention. In particular embodiments, a preferred transposition system is capable of inserting the transposon end in a random or in an almost random manner to 5′-tag and fragment the target nucleic acid.

As used herein, the term “transposition reaction” refers to a reaction wherein one or more transposons are inserted into target nucleic acids, e.g., at random sites or almost random sites. Essential components in a transposition reaction are a transposase and DNA oligonucleotides that exhibit the nucleotide sequences of a transposon, including the transferred transposon sequence and its complement (the non-transferred transposon end sequence) as well as other components needed to form a functional transposition or transposome complex. The DNA oligonucleotides can further comprise additional sequences (e.g., adaptor or primer sequences) as needed or desired. In some embodiments, the method provided herein is exemplified by employing a transposition complex formed by a hyperactive Tn5 transposase and a Tn5-type transposon end (Goryshin and Reznikoff, 1998, J. Biol. Chem., 273:7367) or by a MuA transposase and a Mu transposon end comprising Rland R2 end sequences (Mizuuchi, 1983, Cell, 35:785; Savilahti et al., 1995, EMBO J., 14:4893). However, any transposition system that is capable of inserting a transposon end in a random or in an almost random manner with sufficient efficiency to 5′-tag and fragment a target DNA for its intended purpose can be used in the present invention. Examples of transposition systems known in the art which can be used for the present methods include but are not limited to Staphylococcus aureus Tn552 (Colegio et al., 2001, J Bacterid., 183:2384-8; Kirby et al., 2002, Mol Microbiol, 43:173-86), Tyl (Devine and Boeke, 1994, NucleicAcids Res., 22:3765-72 and International Patent Application No. WO 95/23875), TransposonTn7 (Craig, 1996, Science. 271:1512; Craig, 1996, Review in: Curr Top MicrobiolImmunol, 204:27-48), TnIO and ISIO (Kleckner et al., 1996, Curr Top Microbiol Immunol, 204:49-82), Mariner transposase (Lampe et al., 1996, EMBO J., 15:5470-9), Tci (Plasterk, 1996, Curr Top Microbiol Immunol, 204:125-43), P Element (Gloor, 2004, Methods Mol Biol, 260:97-114), TnJ (Ichikawa and Ohtsubo, 1990, J Biol Chem. 265:18829-32), bacterial insertion sequences (Ohtsubo and Sekine, 1996, Curr. Top. Microbiol. Immunol. 204:1-26), retroviruses (Brown et al., 1989, Proc Natl Acad Sci USA, 86:2525-9), and retrotransposon of yeast (Boeke and Corces, 1989, Annu Rev Microbiol. 43:403-34). The method for inserting a transposon end into a target sequence can be carried out in vitro using any suitable transposon system for which a suitable in vitro transposition system is available or that can be developed based on knowledge in the art. In general, a suitable in vitro transposition system for use in the methods provided herein requires, at a minimum, a transposase enzyme of sufficient purity, sufficient concentration, and sufficient in vitro transposition activity and a transposon end with which the transposase forms a functional complex with the respective transposase that is capable of catalyzing the transposition reaction. Suitable transposase transposon end sequences that can be used in the invention include but are not limited to wild-type, derivative or mutant transposon end sequences that form a complex with a transposase chosen from among a wild-type, derivative or mutant form of the transposase. As used herein, the term “transposome complex” refers to a transposase enzyme non-covalently bound to a double stranded nucleic acid. For example, the complex can be a transposase enzyme pre-incubated with double-stranded transposon DNA under conditions that support non-covalent complex formation. Double-stranded transposon DNA can include, without limitation, Tn5 DNA, a portion of Tn5 DNA, a transposon end composition, a mixture of transposon end compositions or other double-stranded DNAs capable of interacting with a transposase such as the hyperactive Tn5 transposase.

As used herein, the term “random” can be used to refer to the spatial arrangement or composition of locations on a surface. For example, there are at least two types of order for an array described herein, the first relating to the spacing and relative location of features (also called “sites”) and the second relating to identity or predetermined knowledge of the particular species of molecule that is present at a particular feature. Accordingly, features of an array can be randomly spaced such that nearest neighbor features have variable spacing between each other. Alternatively, the spacing between features can be ordered, for example, forming a regular pattern such as a rectilinear grid or hexagonal grid. In another respect, features of an array can be random with respect to the identity or predetermined knowledge of the gene of interest (e.g. nucleic acid of a particular sequence) that occupies each feature independent of whether spacing produces a random pattern or ordered pattern. An array set forth herein can be ordered in one respect and random in another. For example, in some embodiments set forth herein a surface is contacted with a population of nucleic acids under conditions where the nucleic acids attach at sites that are ordered with respect to their relative locations but ‘randomly located’ with respect to knowledge of the sequence for the nucleic acid species present at any particular site. Reference to “randomly distributing” nucleic acids at locations on a surface is intended to refer to the absence of knowledge or absence of predetermination regarding which nucleic acid will be captured at which location (regardless of whether the locations are arranged in an ordered pattern or not).

As used herein, the term “tissue sample” refers to a piece of tissue that has been obtained from a subject, optionally fixed, sectioned, and mounted on a planar surface, e.g., a microscope slide. The tissue sample can be a formalin-fixed paraffin-embedded (FFPE) tissue sample or a fresh tissue sample or a frozen tissue sample, etc. The methods disclosed herein may be performed before or after staining the tissue sample. For example, following hematoxylin and eosin staining, a tissue sample may be spatially analyzed in accordance with the methods as provided herein. A method may include analyzing the histology of the sample (e.g., using hematoxylin and esoins staining) and then spatially analyzing the tissue.

As used herein, the term “formalin-fixed paraffin embedded (FFPE) tissue section” refers to a piece of tissue, e.g., a biopsy that has been obtained from a subject, fixed in formaldehyde (e.g., 3%-5% formaldehyde in phosphate buffered saline) or Bouin solution, embedded in wax, cut into thin sections, and then mounted on a planar surface, e.g., a microscope slide.

As used herein, the term “subject” encompasses mammals and non-mammals. Examples of mammals include, but are not limited to, any member of the mammalian class: humans, non-human primates such as chimpanzees, and other apes and monkey species, cattle, horses, sheep, goats, swine, rabbits, dogs, cats, rodents, rats, mice, guinea pigs, and the like. Examples of non-mammals include, but are not limited to, birds, fish, and the like. The term does not denote a particular age or gender.

In some embodiments, nucleic acids in a tissue sample are transferred to and captured onto an array. For example, a tissue section is placed in contact with an array and nucleic acid is captured onto the array and tagged with a spatial address. The spatially-tagged DNA molecules are released from the array and analyzed, for example, by high throughput next generation sequencing (NGS), such as sequencing-by-synthesis (SBS). In some embodiments, a nucleic acid in a tissue section (e.g., a formalin-fixed paraffin-embedded (FFPE) tissue section) is transferred to an array and captured onto the array by hybridization to a capture probe. In some embodiments, a capture probe can be a universal capture probe hybridizing, e.g., to an adaptor region in a nucleic acid sequencing library, or to the poly-A tail of an mRNA. In some embodiments, the capture probe can be a gene-specific capture probe hybridizing, e.g., to a specifically targeted mRNA or cDNA in a sample, such as a TruSeq™ Custom Amplicon (TSCA) oligonucleotide probe (Illumina, Inc.). A capture probe can be a plurality of capture probes, e.g., a plurality of the same or of different capture probes.

In some embodiments, a combinatorial indexing (addressing) system is used to provide spatial information for analysis of nucleic acids in a tissue sample. The combinatorial indexing system can involve the use of two or more spatial address sequences (e.g., two, three, four, five or more spatial address sequences).

In some embodiments, two spatial address sequences are incorporated into a nucleic acid during preparation of a sequencing library. A first spatial address can be used to define a certain position (i.e., capture site) in the X dimension on a capture array and a second spatial address sequence can be used define a position (i.e., a capture site) in the Y dimension on the capture array. During library sequencing, both X and Y spatial address sequences can be determined and the sequence information can be analyzed to define the specific position on the capture array.

In some embodiments, three spatial address sequences are incorporated into a nucleic acid during preparation of a sequencing library. A first spatial address can be used to define a certain position (i.e., capture site) in the X dimension on a capture array, a second spatial address sequence can be used define a position (i.e., a capture site) in the Y dimension on the capture array, and a third spatial address sequence can be used to define a position of a two-dimensional sample section (e.g., the position of a slice of a tissue sample) in a sample (e.g., a tissue biopsy) to provide positional spatial information in the third dimension (Z dimension) of a sample. During library sequencing, X, Y, and Z spatial address sequences can be determined and the sequence information can be analyzed to define the specific position on the capture array.

In some embodiments, a temporal address sequence (T) is optionally incorporated into a nucleic acid during preparation of a sequencing library. In some embodiments, the temporal address sequence can be combined with two or three spatial address sequences. The temporal address sequence can, for example, be used in the context of a time-course experiment for determining time-dependent changes in gene-expression in a tissue sample. Time-dependent changes in gene-expression can occur in a tissue sample, for example, in response to a chemical, biological or physical stimulus (e.g., a toxin, a drug, or heat). Nucleic acid samples obtained at different timepoints from comparable tissue samples (e.g., proximal slices of a tissue sample) can be pooled and sequenced in bulk. An optional first spatial address can be used to define a certain position (i.e., capture site) in the X dimension on a capture array, a second optional spatial address sequence can be used to define a position (i.e., a capture site) in the Y dimension on the capture array, and a third optional spatial address sequence can be used to define a position of a two-dimensional sample section (e.g., the position of a slice of a tissue sample) in a sample (e.g., a tissue biopsy) to provide positional spatial information in the third dimension (Z dimension) of the sample. During library sequencing, T, X, Y, and Z address sequences are determined and the sequence information is analyzed to define the specific X, Y (and optionally Z) position on the capture array for each timepoint (T).

The address sequences X, Y, and, optionally, Z and/or T, can be consecutive nucleic acid sequences or the address sequences can be separated by one or more nucleic acids (e.g., 2 or more, 3 or more, 10 or more, 30 or more, 100 or more, 300 or more, or 1,000 or more). In some embodiments, the X, Y, and optionally Z and/or T address sequences can each individually and independently be combinatorial nucleic acid sequences.

In some embodiments, the length of the address sequences (e.g., X, Y, Z, or T) can each individually and independently be 100 nucleic acids or less, 90 nucleic acids or less, 80 nucleic acids or less, 70 nucleic acids or less, 60 nucleic acids or less, 50 nucleic acids or less, 40 nucleic acids or less, 30 nucleic acids or less, 20 nucleic acids or less, 15 nucleic acids or less, 10 nucleic acids or less, 8 nucleic acids or less, 6 nucleic acids or less, or 4 nucleic acids or less. The length of two or more address sequences in a nucleic acid can be the same or different. For example, if the length of address sequence X is 10 nucleic acids, the length of address sequence Y can be, e.g., 8 nucleic acids, 10 nucleic acids, or 12 nucleic acids.

Address sequences, e.g., spatial address sequences such as X or Y, can be either partially or fully degenerate sequences.

In some embodiments, spatially addressed capture probes on an array can be released from the array onto a tissue section for generation of a spatially addressed sequencing library. In some embodiments, a capture probe comprises a random primer sequence for in situ synthesis of spatially-tagged cDNA from RNA in the tissue section. In some embodiments, a capture probe is a TruSeq™ Custom Amplicon (TSCA) oligonucleotide probe (Illumina, Inc.) for capturing and spatially tagging genomic DNA in the tissue section. The spatially-tagged nucleic acid molecules (e.g., cDNA or genomic DNA) are recovered from the tissue section and processed in single tube reactions to generate a spatially-tagged amplicon library.

In some embodiments, magnetic nanoparticles can be used to capture nucleic acid (e.g., in situ synthesized cDNA) in a tissue sample for generation of a spatially addressed library.

In some embodiments, spatial detection and analysis of nucleic acid in a tissue sample can be performed on a droplet actuator.

Described herein are improved methods and compositions for spatial-omics applications that preserve spatial information related to the origin of RNA or DNA in the tissue. Examples of spatial omics applications include, but are not limited to, spatial genomic applications, spatial proteomic applications; spatial transcriptomic applications; spatial agrigenomic applications; spatial epigenomics s applications; spatial phenomic applications; spatial ligandomic applications; and spatial multiomic applications (e.g., transcriptomic and genomic applications).

### Preparation of Polynucleotides

The present disclosure is based, in part, on the realization that the amount of RNA or DNA information isolatable from fresh or frozen tissue samples as well as FFPE tissue samples needs to be improved to provide information related to the genetic profile of the tissue sample. The present disclosure provides methods for improved capture of genetic information by increasing the amount and quality of RNA isolated from tissue samples that can be used in spatial transcriptomics analysis. RNA is polyadenylated in situ as described herein, wherein the RNA is contacted with polynucleotide kinase (PNK) to modify 3′ phosphate to a hydroxyl group to generate end repaired total RNA. The end repaired RNA is admixed with polyadenylate polymerase (PAP) and adenosine nucleotides to generate polyadenylated total RNA. The polyA RNA is captured on a substrate comprising oligonucleotides comprising poly T sequences. The oligonucleotides comprising poly T sequences can further comprise capture probe or spatial index sequences, including, but not limited to, one or more of a P7 sequence, an index sequence, and/or a Read 2 (Rd2) sequence.

The total RNA can comprise ribosomal RNA (rRNA), messenger RNA (MRNA), transfer RNA (tRNA), microRNA, small nucleolar RNA (snoRNA), small nuclear RNA (snRNA). In various embodiments, the RNA is rRNA and/or mRNA.

The polyA tails can be between 3 and 50 nucleotides, e.g., from 5-50 nucleotides in length, from 10-40 nucleotides in length, from 15-30 nucleotides in length, or 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In some embodiments, the total RNA is released from the tissue sample after polyadenylation. Release includes lysis of tissue or permeabilization of the tissue. In various embodiments, one or more samples that have been contacted with a solid support can be lysed to release target nucleic acids. Lysis can be carried out using known techniques, such as those that employ one or more of chemical treatment, enzymatic treatment, electroporation, heat, hypotonic treatment, sonication or the like.

In some embodiments, a tissue sample will be treated to remove embedding material (e.g. to remove paraffin or formalin) from the sample prior to release, capture or modification of nucleic acids. This can be achieved by contacting the sample with an appropriate solvent (e.g. xylene and ethanol washes). Treatment can occur prior to contacting the tissue sample with a solid support set forth herein or the treatment can occur while the tissue sample is on the solid support. Exemplary methods for manipulating tissues for use with solid supports to which nucleic acids are attached are set forth in US Pat. App. Publ. No. 2014/0066318, which is incorporated herein by reference.

A formalin-fixed tissue sample may also be decrosslinked using known techniques. In various embodiments, decrosslinking is carried out using Tris-EDTA (TE) buffer, e.g., at pH 8, pH 9, or another appropriate buffer at an appropriate pH. Decrosslinking may also be carried out at high heat, e.g., 70° C.

The present disclosure is further based, in part, on the realization that the capture efficiency of mRNA transcripts for in situ mRNA transcript library preparation can be improved by using high processivity enzymes in either or both first and second strand synthesis reactions. mRNA transcripts isolated from a tissue sample are captured on a substrate and contacted with a high processivity reverse transcriptase (RT) or high processivity DNA polymerase to generate a first cDNA strand complementary to the mRNA transcripts. High processivity RTs include Superscript IV, thermostable group II intron RT (TGIRT), or marathon RT. In various embodiments, the first cDNA strand is contacted with a DNA polymerase to generate a second cDNA strand complementary to the first cDNA strand, optionally the DNA polymerase is a high processivity DNA polymerase. In various embodiments, the high processivity DNA polymerase is Klenow exo−, Bst 3.0, or phi29. In various embodiments, the DNA polymerase lacks both 5→3′ and 3′→5 exonuclease activity.

In some embodiments, mRNA transcripts isolated from a tissue sample are captured on a substrate and contacted with a RT to generate a first cDNA strand complementary to the mRNA transcripts. In various embodiments, the first cDNA strand is contacted with a high processivity RT or high processivity DNA polymerase to generate a second cDNA strand complementary to the first cDNA strand.

In some embodiments, mRNA transcripts isolated from a tissue sample are captured on a substrate and contacted with a high processivity RT or high processivity DNA polymerase having RT activity to generate a first cDNA strand complementary to the mRNA transcripts. In various embodiments, the first cDNA strand is contacted with a high processivity RT or high processivity DNA polymerase to generate a second cDNA strand complementary to the first cDNA strand.

In some embodiments, mRNA transcripts isolated from a tissue sample are captured on a substrate and contacted with a high processivity RT to generate a first cDNA strand complementary to the mRNA transcripts. In various embodiments, the first cDNA strand is contacted with a high processivity RT or high processivity DNA polymerase to generate a second cDNA strand complementary to the first cDNA strand.

After second strand synthesis the second strand cDNA is amplified to form a PCR template and the PCR template is isolated using standard techniques.

The methods above are also useful for improving capture efficiency of mRNA transcripts for in situ mRNA transcript library preparation, and/or for improving the nucleotide length of polynucleotides used in generating an in situ transcriptome library (e.g., improving the polynucleotide size of cDNA transcribed from mRNA isolated from a sample and used in generating an in situ transcriptome library)

The present disclosure is further based, in part, on the realization that the in situ polyadenylation method described herein can be used in combination with the high processivity enzymes for first and or second strand synthesis to improve spatial transcriptomics RNA library preparation. For example, the disclosure provides a method for preparing an mRNA transcriptome library from a tissue sample comprising, contacting total RNA isolated from the sample with polynucleotide kinase (PNK) to modify 3′ phosphate to a hydroxyl group to generate end repaired total RNA; contacting the end repaired total RNA with polyadenylate polymerase (PAP) and adenosine nucleotides to generate polyadenylated total RNA; releasing the polyadenylated total RNA from the tissue sample; capturing the polyadenylated total RNA on a substrate comprising one or more oligonucleotides comprising a poly T sequence; depleting ribosomal RNA from the total RNA leaving polyadenylated mRNA; eluting the polyadenylated mRNA from the substrate; contacting the polyadenylated mRNA with a high processivity reverse transcriptase (RT) to generate a first cDNA strand complementary to the mRNA transcripts; contacting the first cDNA strand with the high processivity reverse transcriptase (RT) or high processivity DNA polymerase to generate a second cDNA strand complementary to the first cDNA strand to generate PCR templates; eluting the PCR templates; and generating an mRNA library from the PCR templates using methods described herein.

### Spatial Detection and Analysis of Nucleic Acids in a Tissue Sample

According to the methods described herein, spatial detection and analysis of nucleic acids in a tissue sample can be performed using sets of two or more capture probes (e.g., 3 or more, 4 or more, 5 or more, 6 or more, 7 or more, 8 or more, 9 or more, or 10 or more capture probes). Typically at least a first capture probe in a set of capture probes is immobilized on a capture array. In some embodiments, a second capture probe can be immobilized on the same capture array as the first capture probe, e.g., in proximity to the first capture probe, e.g., in the same capture site. In some embodiments, a second capture probe can be immobilized on a particle, such as a magnetic particle or a magnetic nanoparticle. In some embodiments, a second capture probe can be in solution, e.g., to be used to perform in situ reactions with a nucleic acid in a tissue sample. The capture probes in the capture probe sets individually and independently can have a variety of different regions, e.g., a capture region (e.g., a first universal or gene-specific capture region or first clustering region), a primer binding region (e.g., a SBS primer region, such as a SBS3 or SBS12 region), or a second universal region/clustering sequence, such as a P5 or P7 region, a spatial address region (e.g., a partial or combinatorial spatial address region), or a cleavable region.

Exemplary sequences include the following Rd1 and Rd2 adaptor sequences.

Second Universal Adapter-Rd1 SBS3 (long):
(SEQ ID NO: 7)
ACACTCTTTCCCTACACGACGCTCTTCCGATCT;
Second Universal Adapter-Rd1 SBS3 (short):
(SEQ ID NO: 8)
ACACTCTTTCCCTACACGAC;
First Universal Adapter-Rd2 SBS12 (long):
(SEQ ID NO: 9)
GTGACTGGAGTTCAGACGTGTGCTCTTCCGATCT;
First Universal Adapter-Rd2 SBS12 (short):
(SEQ ID NO: 10)
GTGACTGGAGTTCAGACGTGT.

In some embodiments, only one capture probe in a set of capture probes comprises a capture region. In some embodiments, two or more capture probes in a set of capture probes comprise as capture region.

In some embodiments, only one probe in a set of capture probes comprises a spatial address region, e.g., such as a complete spatial address region describing the position of a capture site on a capture array. In some embodiments, two or more probes in a set of capture probes can comprise a spatial address region, e.g., two or more probes can each comprise a partial spatial address region (i.e., combinatorial address region), wherein each partial address region describes the position of a capture site on a capture array, e.g., along the x-axis or the y-axis.

In some embodiments, a set of capture probes (e.g., a first and second capture probe) can comprise at least one capture probe comprising a capture region and a spatial address region (e.g., a complete or a partial spatial address region). In some embodiments, no capture probe in a set of capture probes comprises both a capture region and a spatial address region.

In some embodiments, the first capture probe is a 5′ gene specific probe comprising a sequence complementary to the first universal adapter sequence and a 5′ gene specific primer.

In some embodiments, the second capture probe is a 3′ gene specific probe comprising a 3′ gene specific primer, a unique molecular index (UMI), and a second universal adapter sequence (Rd1 adapter). In some embodiments, the second capture probe does not comprise a spatial address region.

In some embodiments, the capture site on the substrate is a plurality of capture sites. In some embodiments, the plurality of capture sites is 2 or more, 10 or more, 30 or more, 100 or more, 300 or more, 1,000 or more, 3,000 or more, 10,000 or more, 30,000 or more, 100,000 or more, 300,000 or more, 1,000,000 or more 3,000,000 or more, or 10,000,000 or 1,000,000,000 or more capture sites.

In various embodiments, the capture array or substrate comprises a capture site density of 1 or more, 2 or more, 10 or more, 30 or more, 100 or more, 300 or more, 1,000 or more, 3,000 or more, 10,000 or more, 100,000 or more, 1,000,000 or more, capture sites per square centimeter (cm²).

In various embodiments, the pair of capture probes in a capture site is a plurality of pairs of capture probes. In some embodiments, the plurality of capture probes is 2 or more, 10 or more, 30 or more, 100 or more, 300 or more, 1,000 or more, 3,000 or more, 10,000 or more, 30,000 or more, 100,000 or more, 300,000 or more, 1,000,000 or more 3,000,000 or more, or 10,000,000 or more, 100,000,000 or more, or 1,000,000,000 or more capture probes.

In some embodiments, the pair of capture probes in a capture site of a substrate is a plurality of pairs of capture probes. In some embodiments, each first capture probe in the plurality of pairs of capture probes within the same capture site comprises the same spatial address sequence. In some embodiments, each first capture probe in the plurality of pairs of capture probes in different capture sites comprises a different spatial address sequence.

In some embodiments, the surface of the capture array is a planar surface, e.g., a glass surface. In some embodiments, the surface of the capture array comprises one or more wells. In some embodiments, the one or more wells correspond to one or more capture sites. In some embodiments, the surface of the capture array is a bead surface.

In some embodiments, the capture region in the second capture probe is a gene-specific capture region. In some embodiments, the gene-specific capture region in the second capture probe comprises the sequence of a TruSeq™ Custom Amplicon (TSCA) oligonucleotide probe (Illumina, Inc.). For example, the gene-specific capture regions in a plurality of second capture probes in a capture site can comprise a plurality of sequences of TSCA oligonucleotide probes.

In another embodiment, the disclosure provides for nanoparticles or beads which comprise the spatially addressable probes disclosed herein. In a particular embodiment, beads comprise the spatially addressable probes disclosed herein. In a further embodiment, the bead comprises streptavidin on the surface of the bead. In yet a further embodiment, the beads comprise a plurality of oligos bound to the bead via a linkage or a reversible linkage. Examples of reversible linkages include biotin molecule(s), such as ddBio molecules. The oligos bound the bead typically comprise an adaptor sequence, such as P5 sequence or a P7 sequence. As used herein a P5 sequence comprises a sequence defined by AAT GAT ACG GCG ACC ACC GA (SEQ ID NO: 1) or AAT GAT ACG GOG ACC ACC GAG ATC TAC AC (SEQ ID NO: 2) and a P7 sequence comprises a sequence defined by CAA GCA GAA GAC GGC ATA CG (SEQ ID NO: 3) or CAA GCA GAA GAC GGC ATA CGA GAT (SEQ ID NO: 4). In some embodiments, the P5 or P7 sequence can further include a spacer polynucleotide, which may be from 1 to 20, such as 1 to 15, or 1 to 10, nucleotides, such as 2, 3, 4, 5, 6, 7, 8, 9, or 10 nucleotides in length. In some embodiments, the spacer includes 10 nucleotides. In some embodiments, the spacer includes 10 nucleotides. In some embodiments, the spacer is a polyT spacer, such as a 10T spacer. Spacer nucleotides may be included at the 5′ ends of polynucleotides, which may be attached to a suitable support via a linkage with the 5′ end of the oligo. Attachment can be achieved through a sulfur-containing nucleophile, such as phosphorothioate, present at the 5′ end of the polynucleotide. In some embodiments, the oligos will include a polyT spacer and a 5′phosphorothioate group. Thus, in some embodiments, the P5 sequence comprises 5′phosphorothioate-TTTTTTTTTTAATGATACGGCGACCACCGA-3′ (SEQ ID NO: 5), and in some embodiments, the P7 sequence comprises 5′ phosphorothioate-TTTTTTTTTTCAAGCAGAAGACGGCATACGA-3′ (SEQ ID NO: 6). In certain embodiments, the oligos attached to the beads comprise an address sequence that allows for determining the x, y position of the oligo/bead when decoded. In further embodiments, the address sequence is 5, 10, 15, 20, 25, 30, 35, 40, 45, 50, 55, 60, 65, 70, 75, 80, 85, 90, 95, or 100 nucleotides in length, or a range that includes or is between any two of the foregoing nucleotides in length. In another embodiment, the oligos attached to the beads comprise a transposome hybridization region (Tsm hyb). In yet additional embodiments, the oligos comprise sequencing primer(s) site sequence(s). Examples of sequencing primer site sequences include sequences that are complementary to R1 and R2 sequencing primers from Illumina™. In further embodiments, the oligos may further comprise one or more linker sequences. In yet further embodiment, the oligos may further comprise one or more index sequences. In certain embodiments, the oligos may comprise one or more unique molecular identifier (UMI) sequences. Unique molecular identifiers (UMIs) are a type of molecular barcoding that provides error correction and increased accuracy during sequencing. These molecular barcodes are short sequences used to uniquely tag each molecule in a sample library. UMIs are used for a wide range of sequencing applications, many around PCR duplicates in DNA and cDNA. UMI deduplication is also useful for RNA-seq gene expression analysis and other quantitative sequencing methods. As noted previously, the oligos comprise moieties or sequences that can bind with specificity to polynucleotides from a biological sample (e.g., a tissue sample). As such, the oligos attached to the beads are spatially addressable probes for polynucleotides from a biological sample. The moieties or sequences that can bind with specificity to polynucleotides from a biological sample can be selected for a particular omic application. For example, the oligos can comprise an oligo d (T) sequence for transcriptomics or for assay (e.g., RNA-seq assays). Alternatively, the oligos can comprise sequences to bind with genomic DNA from a biological sample for genomic applications or for assays (e.g., ATAC-seq assays). As provided in the Examples presented herein, the beads can comprise multiple types of oligos that have different moieties or sequences so that the spatially addressable probes can bind specifically to two or more different types of polynucleotides from a biological sample. The use of multi types of oligos is ideally suited for multiomic or multiple assay applications.

### Kits

Kits and articles of manufacture are also contemplated herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic. For example, the container(s) can comprise one or more spatially addressable probes disclosed herein, optionally in a composition or in combination with another agent (e.g., an array, a beadchip) as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use with the spatially addressable probes described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific spatial omic applications. The label can also indicate directions for use of the contents, such as in the methods described herein.

The following examples are intended to illustrate but not limit the disclosure. While they are typical of those that might be used, other procedures known to those skilled in the art may alternatively be used.

### EXAMPLES

### Example 1—In Situ Capture of FFPE RNA for Spatial Transcriptomics Applications

The genetic profile of a tissue sample may be used to diagnose and determine treatment for a subject having or at risk of having a disease as determined by the genetic profile. In situ polyadenylation is explored as a method to increase capture of RNA from FFPE tissue samples. In situ polyadenylation adds polyA tails to fragmented transcripts, generating regions that are then available for capture on polyT surface.

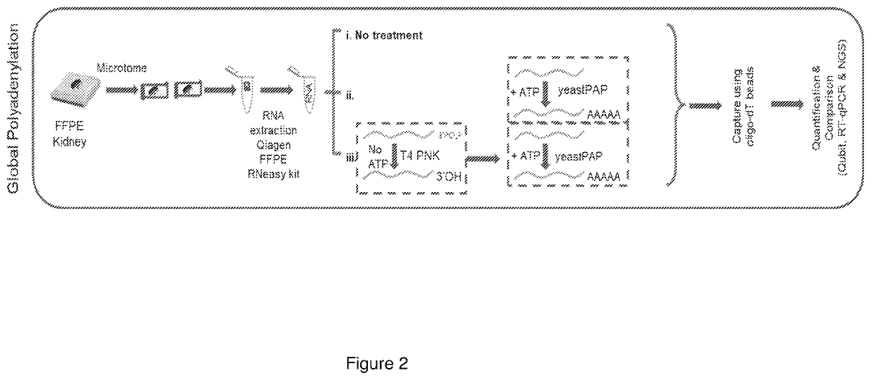

Total RNA was extracted and used for this initial experiment. Three conditions were tested: 1.) No treatment, 2.) Polyadenylation, and 3.) End repair with polynucleotide kinase (PNK), which converts 3′ phosphates to hydroxyls for polyA addition, followed by polyadenylation. (FIG. 2)

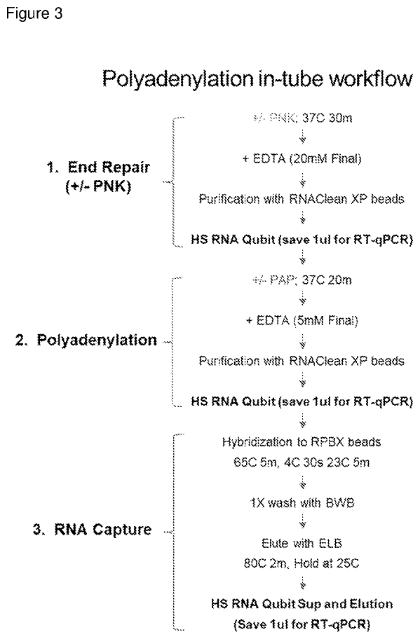

In an initial experiment (FIG. 3), total RNA was extracted from FFPE tissue using RNeasy FFPE Kit (Qiagen). Total RNA was extracted from fresh frozen tissue with RNeasy Mini Kit (Qiagen). 500 ng total RNA was used for each condition. Samples were treated with +/−end repair polynucleotide kinase (PNK) mix (1×T4 PNK Buffer (NEB), 10U T4 PNK (NEB), water), at 37° C. for 30 minutes. The reaction was then stopped with 20 mM final EDTA. Samples were purified with RNAClean XP beads (1.8× reaction volume). Samples were then treated with +/−PAP mix (1×yPAP reaction buffer (ThermoFisher), 500 uM ATP, 600U yPAP, water) and incubated at 37° C. for 20 minutes. The reaction was stopped with 5 mM final EDTA. Samples were purified with RNAClean XP beads (1.8× reaction volume). Samples were then hybridized to Illumina RPBX oligo-dT beads following these parameters: 65° C. 5 minutes, 4° C. 30 seconds, and 23° C. 5 minutes. Samples were washed once with Illumina bead wash buffer (BWB). Samples were then eluted in Illumina elution buffer (ELB). Elutions were quantified with high sensitivity RNA QUBIT® Kit.

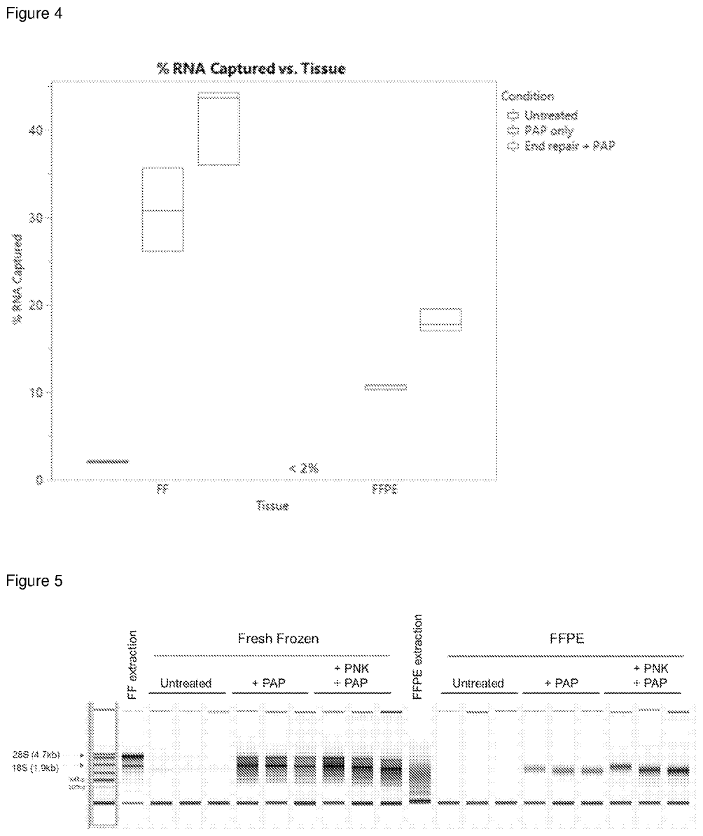

QUBIT concentrations were plotted between conditions (FIG. 4). Oligo-dT beads are saturated after PAP, suggesting increased capture of polyA RNA. PAP significantly improves capture of polyA RNA for FFPE and fresh frozen samples. End repair improves capture ˜10%. The expected capture of ˜2% mRNA in untreated samples is observed (which makes up 1-5% of total RNA). TapeStation analysis shows capture of 18S and 28S (non-polyA RNAs) from total RNA population, suggesting the polyadenylation is working to improve RNA capture (FIG. 5).

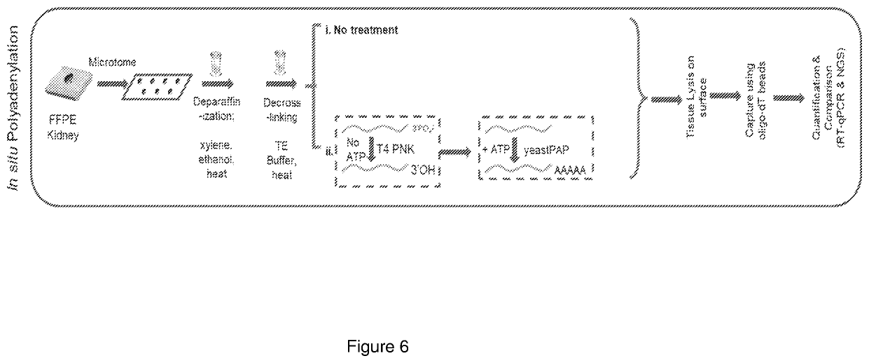

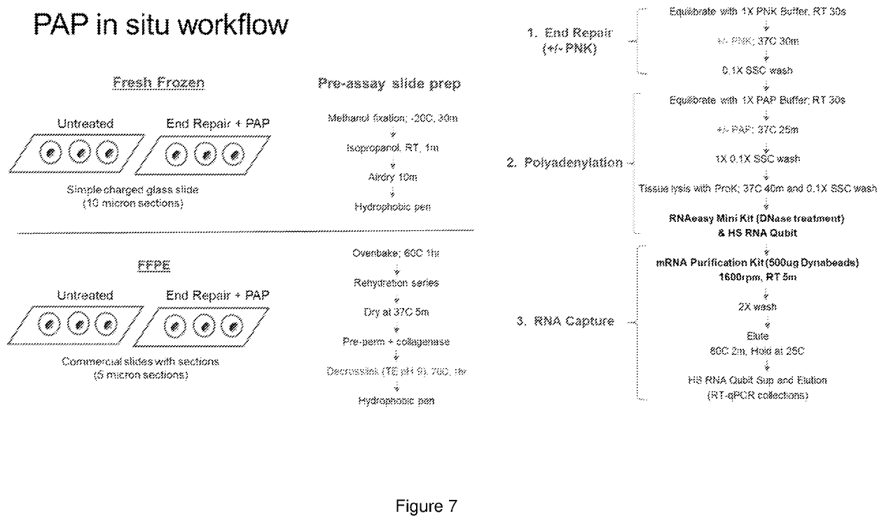

To test PAP in situ, a similar protocol to FIG. 2 was designed (FIG. 6). In this experiment end repair was added for all PAP samples (FIG. 7). Three 10 micron fresh frozen tissue sections were fixed on two charged glass slides (one slide for untreated tissue, one slide for polyadenylation). The slides were fixed in methanol at −20° C. for 30 minutes, treated with isopropanol at room temperature for 1 minute, and then air-dried for 10 minutes. A hydrophobic pen was then used to draw a barrier around each fresh frozen tissue section. Commercial FFPE tissue with fixed tissue sections were obtained from Zyagen. Slides were ovenbaked at 60° C. for 1 hour, followed by a rehydration series as follows: two incubations in xylene for 10 minutes at room temperature, followed by three 3 minute incubations in 100% ethanol, followed by two 3 minute incubations in 96% ethanol, followed by a 3 minute incubation in 70% ethanol. The surface was then treated with nuclease-free water for 1 minute at room temperature. The slides were then dried at 37° C. for 5 minutes. Pre-permeabilization mix was then added to the tissue (986 ul HBS buffer (Life tech), 10 ul BSA (20 mg/ml), 4 ul collagenase I (50U/μl, Life tech)) and incubated at 37° C. for 20 minutes. The pre-permeabilization mix was then removed and TE (pH 9) is added to the slide to decrosslink. A hydrophobic pen was then used to draw a barrier around each FFPE tissue. Samples were treated with +/−end repair PNK mix (1×T4 PNK Buffer (NEB), 10U T4 PNK (NEB), 40U Protector RNase Inhibitor (Millipore Sigma), water), at 37° C. for 30 minutes. The samples were then washed with 100 ul 0.1×SSC buffer. Each well was then equilibrated with 100 ul 1×yPAP buffer (1×yPAP reaction buffer, 40U Protector RNase Inhibitor, water) at room temperature for 30 seconds. The solution was discarded and tissue was incubated in 75 ul yPAP reaction mix (1×yPAP reaction buffer, 1 nM ATP, 600U PAP, 120U Protector RNase Inhibitor, water) for 25 minutes at 37° C. The PAP mix was discarded, and the tissue was washed one time with 100 ul 0.1×SSC. 60 ul of tissue digestion mix (100 mM Tris Buffer, pH 8, 100 mM NaCl, 5 mM EDTA, 2% SDS, 16U/ml Proteinase K (NEB)), was then add to the tissue and incubated at 37° C. for 40 minutes. The tissue digestion mix was then removed and added to strip tubes. An additional wash with 50 ul 0.1×SSC was performed within the barrier and added to the sample's tissue digestion mix. All samples were then purified with RNeasy Mini Kit (Qiagen) with a DNase step, according to manufacturer's instructions. Samples were quantified with high sensitivity RNA QUBIT kit and 50 ng was set aside for “non-captured” control. Remaining RNA was subject to Invitrogen's mRNA Purification Kit (oligo-dT beads), according to manufacturer's instructions. Captured RNA was quantified by RT-qPCR and high sensitivity RNA QUBIT® Kit.

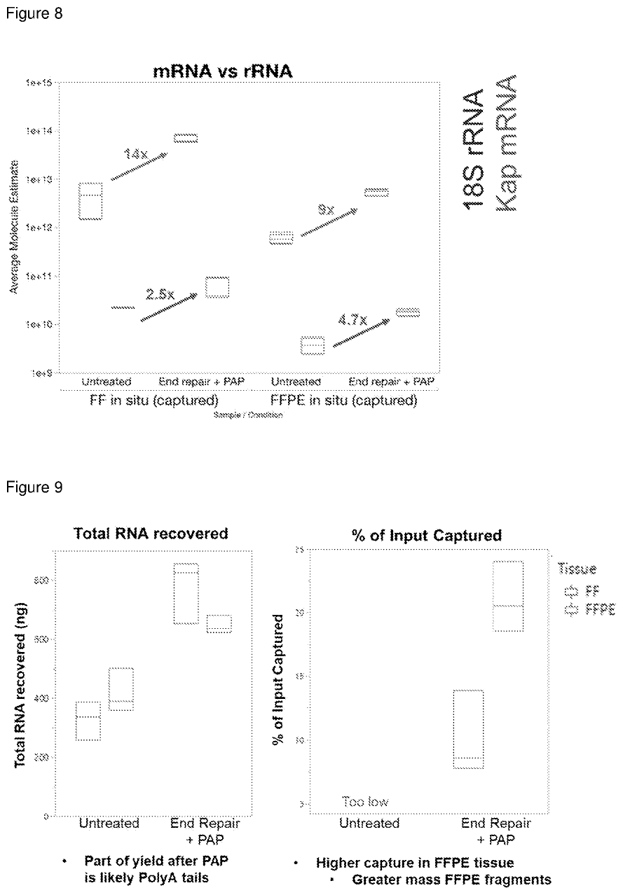

Primer and probe pairs were designed against Kap (mRNA) and 18S (rRNA) to study fold differences with polyadenylation. Primer/probe pairs were used with QuantiNova RT-qPCR kit (Qiagen) according to manufacturer's instructions. Including end-repair with PNK and polyadenylation in workflow generates more polyA transcripts, which generates more capture of mRNA (4.7-fold increase for Kap in FFPE tissue) and rRNA (9-fold increase for 18S in FFPE tissue) (FIG. 8). Captured RNA was also quantified with QUBIT. Including end-repair and polyadenylation to the sample preparation increases capture of RNA from fresh frozen and FFPE tissue, suggesting in situ polyadenylation is working efficiently (FIG. 9).

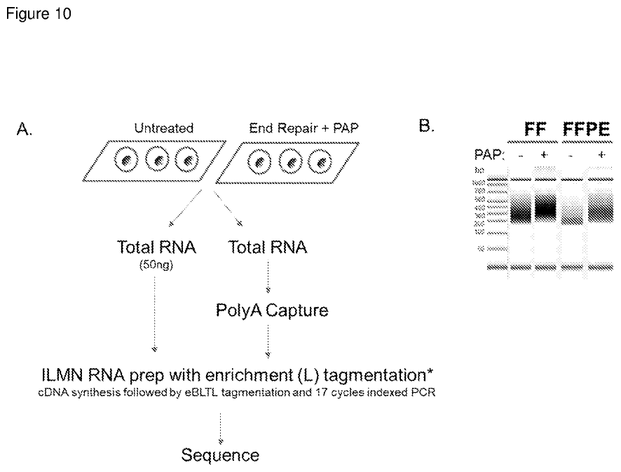

RNA-Seq libraries were prepared following Illumina's RNA Prep with Enrichment (L) tagmentation (without enrichment step) (FIG. 10A). This library prep uses low density eBLTLs for transposition to fragment library and add PCR adapters. 17 cycles of indexed PCR using UD indices was used. TapeStation shows that polyadenylation increases library fragment size. Libraries were normalized, pooled, and 0.8 pM was sequenced on a Nextseq with 1% PhiX (FIG. 10B).

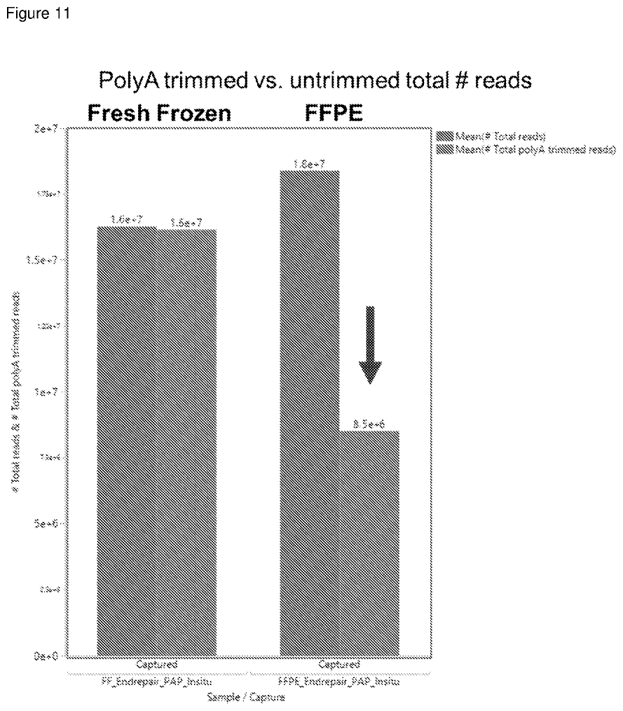

PolyA trimming of fastq libraries shows that ˜50% of FFPE PAP-treated library yield is transposed polyA. This suggests that polyadenylation is working. Additionally, the length of polyA tail addition can be controlled by a ligation approach. RNA ligase 2 Deletion Mutant (as used in Illumina's small RNA prep kit; Epicentre), can ligate polyA adapters to 3′ ends of transcripts for enrichment on a polyT surface (FIG. 11).

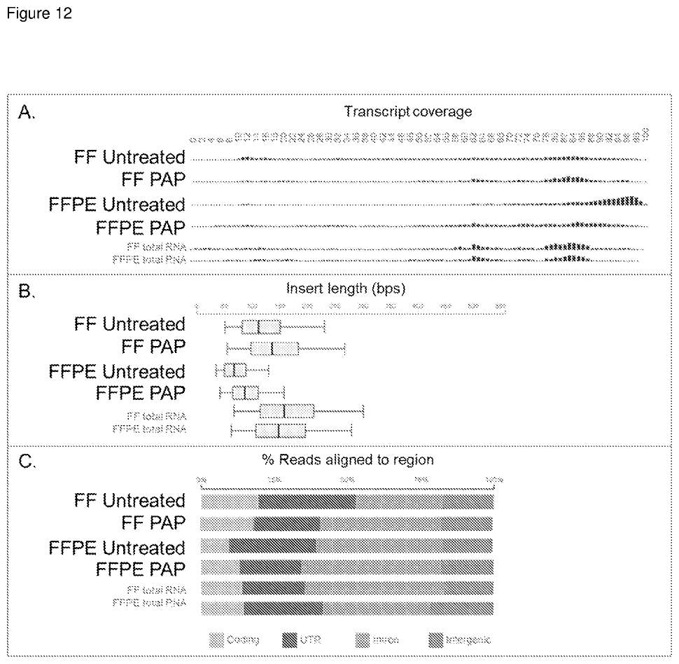

Isolated sequences were aligned using the basespace RNA-seq alignment app. Alignment analysis shows that polyadenylation shifts 3′ bias transcript coverage (FIG. 12A) and increases insert size (FIG. 12B). Polyadenylation also increases % reads aligning to coding regions for FFPE isolated mRNA (FIG. 12C). These sequencing metrics suggest in situ polyadenylation is effective and could serve as a method to increase capture of FFPE RNA on a spatial, barcoded substrate.

### Example 2—Use of High Processivity Polymerases for mRNA Library Preparation

Traditionally, reverse transcriptase (RT) was achieved in 1-step or 2-step workflow with different types of polymerases for each step depending on the template type (ssDNA or ssRNA) and priming strategy (oligo-dT, randomer, or both combined). In a 2-step workflow that uses oligo-dT as the primer in the first strand RT synthesis and randomer as the primer in the second strand synthesis 3′-bias is expected in aligned transcript coverage in RNA-seq applications. The optimized general practice that has been established previously is to use maxima H−, the well-known RTase that has been assessed as having the highest efficiency (as in cDNA yield, not in quality) for the first strand RT synthesis, and then follow-up for the second strand synthesis with a common DNA polymerase that has some strand displacement activity (such as Klenow fragment exo−).

This approach has encountered issues for its inefficiencies in synthesizing good quality cDNA from permeabilized tissue samples on a grafted flow cell (FC) surface that may be attributed to mRNA degradation before and/or during RT as well as low RT efficiency on the FC surface. Subsequent sequencing on the cDNA region presented the symptom of high poly A % fraction, up to >60% in Base % indicating short and/or fragmented cDNA synthesized.

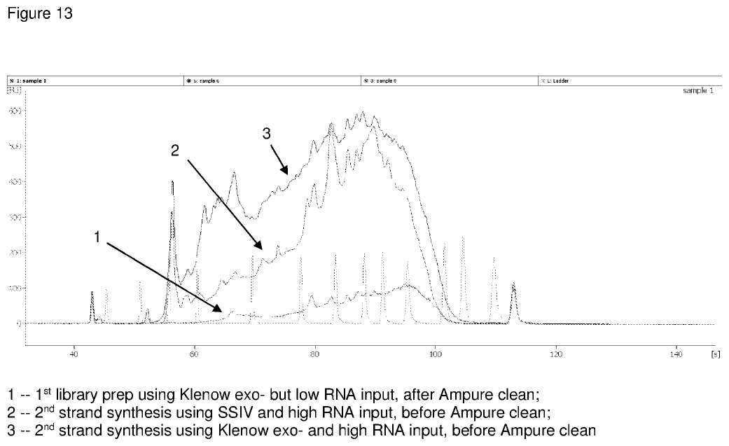

Multiple methods were tested herein to improve the cDNA data quality, and after switching polymerase from Klenow fragment exo− to SSIV in secondary strand synthesis a higher portion of cDNA was observed in the 400-1000 bp region in SSIV conditions comparing to the Klenow exo-control (FIG. 13).

Overall, there are several benefits of using a faster processivity enzyme like SSIV in the 2-step workflow when it applies to Spatial Genomics library preparation. When using SSIV as the RTase for 1st strand RT synthesis, it shortens the workflow time from a 16-20 hour overnight incubation down to 1 hr, with comparable hands-on time. This not only saves time and improves workflow efficiency, but also reduces the concern of mRNA diffusion during capturing, which is a major limiting factor for spatial resolution.

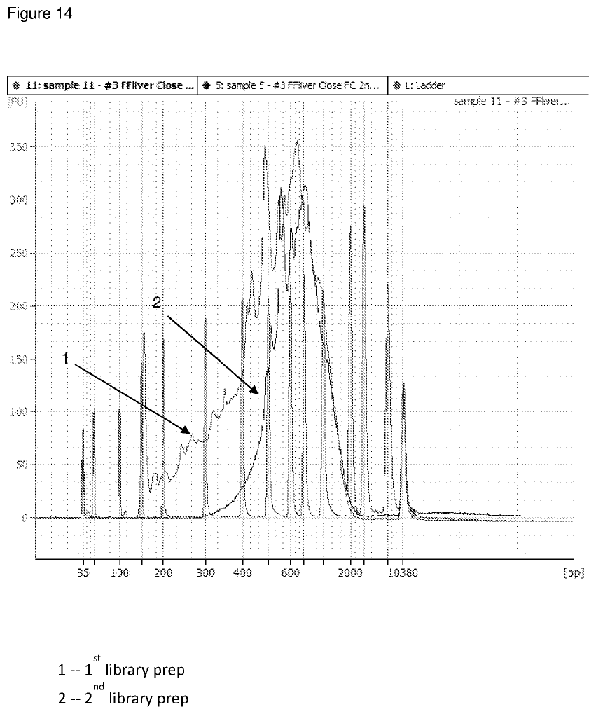

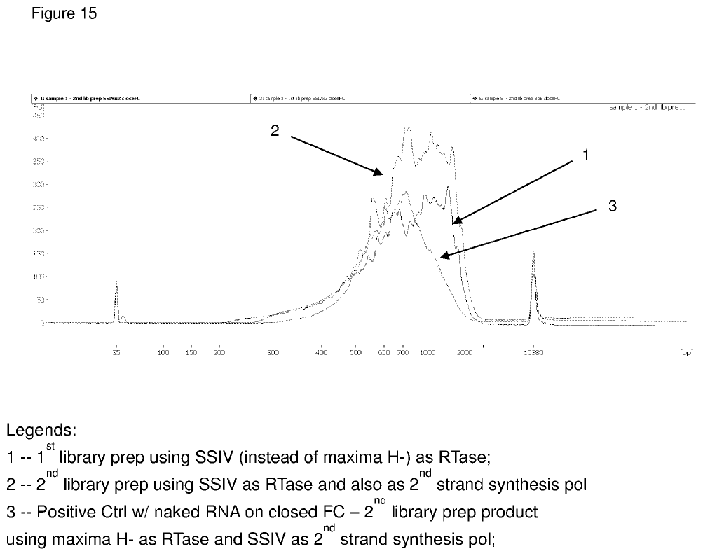

Using SSIV as the polymerase for 2nd strand synthesis, though against the mainstream general practice, in fact improves the easiness for SPRI selection in subsequent library prep, because it preferentially improved the cDNA length to 400-1000 bp, leaving the portion<400 bp mRNA relatively lower comparing to the main peak and thus easier to clean up (FIG. 14). Additionally, using SSIV as either RTase for 1st strand RT synthesis in addition to being the polymerase for 2nd strand synthesis further improves the length and thus the intactness (and likely complexity) of the cDNA in the final product (FIG. 15).

Subsequent data analysis from RNA-seq alignment showed that using SSIV as both RTase for 1st strand RT synthesis or polymerase for 2nd strand synthesis results in highest mapping portion for exonic region, in particular in the coding region (captured coding region increased from −52% to −62%), with reduced 3′ bias (which is due to the polydT priming strategy) median CV for transcript coverage was reduced from −1.41 to 1.34 (Table 1 and Table 2)

TABLE 1
RNA-seq alignment with maxima H- as RTase in 1^{st}
strand synthesis, SSIV as pol in 2^{nd} strand synthesis
Alignment Information

| Region | Fold Coverage | % Bases |
|---|---|---|
| Coding | 17.50x | 51.92% |
| UTR | 11.15x | 35.27% |
| Intron | 0.08x | 6.51% |
| Intergenic | 0.04x | 6.30% |

TABLE 2
RNA-seq alignment SSIVx2 (SSIV as both 1^{st}
strand RTase and 2^{nd} strand synthesis)
Alignment Information

| Region | Fold Coverage | % Bases |
|---|---|---|
| Coding | 23.31x | 62.01% |
| UTR | 9.09x | 25.79% |
| Intron | 0.07x | 5.39% |
| Intergenic | 0.05x | 6.82% |

In general practice, though SSIV is an RTase and could bind to both ssDNA and ssRNA, it has generally not been recommended for use in 2nd strand synthesis because it requires ssDNA as template, while RTase generally binds to RNA more preferentially than ssDNA. Also investigators tend to use one RTase instead of 2 RTases for the workflow. Another previous concern for using SSIV is the relatively lower fidelity in RTase comparing to normal DNA polymerase partially due to their loss in proofreading function and partially due to the dual template use of both DNA and RNA. However, the adaptation of SSIV herein has not revealed any mutation concern in downstream RNA-seq alignment analysis.

Unexpectedly, the reduction of 3′ bias in median CV for transcript coverage (down from −1.41 to 1.34 for test in a closed FC) in RNA-seq alignment analysis results when using SSIV as both RTase for 1st strand RT synthesis and polymerase for 2nd strand synthesis is beneficial and could be further explored.

The results show the methods herein provide clear improvement in isolating longer cDNA fragments in the range of 500-3000 bp as a peak shift on Bioanalyzer, but also much lower polyA percentage (−30%), with improved transcript alignment reaching >89% in RNA-seq alignment (STAR aligner), over 90% of which are exonic transcripts. In particular, using SSIV in both 1^{st} and 2nd strand synthesis increases coding region percentage in mapped transcripts from −52% to 62% while reducing 3′ bias in CV of transcript coverage. The adoption of SSIV as 2nd strand synthesis polymerase contributes critically to the delivery of the improved mRNA transcripts and library preparation.

It is understood, therefore, that this invention is not limited to the particular embodiments disclosed, but is intended to cover all modifications which are within the spirit and scope of the invention as defined by the appended claims; the above description, and/or shown in the attached drawings. Consequently only such limitations as appear in the appended claims should be placed on the disclosure.

What is claimed is:

1. A method for isolating RNA from a sample comprising,
(a) contacting total RNA isolated from the sample with polynucleotide kinase (PNK) to modify 3′ phosphate to a hydroxyl group to generate end repaired total RNA;
(b) contacting the end repaired total RNA with polyadenylate polymerase (PAP) and adenosine nucleotides to generate polyadenylated total RNA;
(c) capturing the polyadenylated total RNA on a substrate comprising one or more oligonucleotides comprising poly T sequences; and
(d) eluting the polyadenylated total RNA from the substrate.

2. The method of claim 1, further comprising quantifying the total RNA.

3. A method for preparing an RNA library from a tissue sample comprising,
(a) contacting total RNA isolated from the sample with polynucleotide kinase (PNK) to modify 3′ phosphate to a hydroxyl group to generate end repaired total RNA;
(b) contacting the end repaired total RNA with polyadenylate polymerase (PAP) and adenosine nucleotides to generate polyadenylated total RNA;
(c) releasing the polyadenylated total RNA from the tissue sample;
(d) capturing the polyadenylated total RNA on a substrate comprising one or more oligonucleotides comprising a poly T sequence; and
(e) generating an RNA library from the polyadenylated total RNA using a RNA library prep kit.

4. The method of any one of claims 1 to 3, wherein the RNA comprises rRNA and/or mRNA.

5. A method for preparing an mRNA transcriptome library from a tissue sample comprising,
(a) contacting total RNA isolated from the sample with polynucleotide kinase (PNK) to modify 3′ phosphate to a hydroxyl group to generate end repaired total RNA;
(b) contacting the end repaired total RNA with polyadenylate polymerase (PAP) and adenosine nucleotides to generate polyadenylated total RNA;
(c) releasing the polyadenylated total RNA from the tissue sample;
(d) capturing the polyadenylated total RNA on a substrate comprising one or more oligonucleotides comprising a poly T sequence;
(e) depleting ribosomal RNA from the total RNA to leave polyadenylated mRNA; and
(f) generating an mRNA library from the polyadenylated mRNA using a mRNA library prep kit.

6. The method of any one of the preceding claims, wherein the substrate is a bead, a bead array, a spotted array, a flow cell, clustered particles arranged on a surface of a chip, a film, and a plate.

7. The method of any one of the preceding claims, wherein the sample is a fresh frozen tissue sample or a formalin-fixed paraffin embedded (FFPE) sample.

8. The method of any one of claims 3 to 7, wherein releasing comprises contacting the sample with a lysis buffer, a pemeabilization buffer and/or a reagent to deparaffinize a FFPE sample.

9. The method of any one of claims 3 to 8, wherein when the sample is a FFPE sample on a slide, the method comprises permeabilization and collagenase treatment of the sample on the slide prior to contacting the RNA with PNK.

10. The method of any one of claims 7 to 9, further comprising decrosslinking the FFPE sample, optionally wherein the decrosslinking is carried out using TE buffer, pH 9.

11. The method of any one of the preceding claims, wherein the polyA tail is between 3 and 50 nucleotides.

12. The method of any one of claims 3 to 11, wherein generating the RNA library comprises the steps of eluting the polyadenylated total RNA from the substrate and generating the RNA library from the eluted polyadenylated RNA library using a RNA library prep kit.

13. The method of any one of claims 3 to 12, wherein generating the RNA library comprises,
i) contacting the isolated RNA with a reverse transcriptase (RT) to generate a first cDNA strand complementary to the RNA;
ii) contacting the first cDNA strand with a reverse transcriptase (RT) or DNA polymerase to generate a second cDNA strand complementary to the first cDNA strand;
iii) amplifying the second strand cDNA to form a PCR template and isolating the PCR template; and
iv) generating an RNA library from the PCR templates.

14. The method of any one of claims 3 to 13, wherein the RNA library is an mRNA library.

15. The method of claim 14, wherein the PCR templates are further processed by tagmentation to generate a spatial transcriptomics library.

16. The method of claim 15 wherein the tagmentation comprises on bead tagmentation, wherein the bead comprises a plurality of bead-linked transposomes (BLT).

17. The method of claim 16, wherein the BLT comprises
i) a plurality of oligonucleotides comprising a first clustering sequence (P7), a first index sequence and a Read 1 sequencing primer (Rd1 SP); and
ii) a plurality of oligonucleotides comprising a second clustering sequence (P5), a second index sequence and a Read 2 sequencing primer (Rd2 SP).

18. A method for improving capture efficiency of mRNA transcripts for in situ mRNA transcript library preparation comprising,
(a) capturing mRNA transcripts from a sample on a substrate;
(b) contacting the substrate with a high processivity reverse transcriptase (RT) to generate a first cDNA strand complementary to the mRNA transcripts;
(c) contacting the first cDNA strand with a DNA polymerase to generate a second cDNA strand complementary to the first cDNA strand; and
(d) amplifying the second strand cDNA to form a PCR template and isolating the PCR template.

19. A method for improving capture efficiency of mRNA transcripts for in situ mRNA transcript library preparation comprising,
(a) capturing mRNA transcripts from a sample on a substrate;
(b) contacting the substrate with a reverse transcriptase (RT) to generate a first cDNA strand complementary to the mRNA transcripts;
(c) contacting the first cDNA strand with a high processivity reverse transcriptase (RT) or high processivity DNA polymerase to generate a second cDNA strand complementary to the first cDNA strand; and
(d) amplifying the second strand cDNA to form a PCR template and isolating the PCR template.

20. A method for improving capture efficiency of mRNA transcripts for in situ mRNA transcript library preparation comprising,
(a) capturing mRNA transcripts from a sample on a substrate;
(b) contacting the substrate with a high processivity reverse transcriptase (RT) to generate a first cDNA strand complementary to the mRNA transcripts;
(c) contacting the first cDNA strand with the high processivity reverse transcriptase (RT) or high processivity DNA polymerase to generate a second cDNA strand complementary to the first cDNA strand; and
(d) amplifying the second strand cDNA to form a PCR template and isolating the PCR template.

21. A method for improving the nucleotide length of polynucleotides used in generating an in situ transcriptome library comprising,
(a) capturing mRNA transcripts from a sample on a substrate;
(b) contacting the substrate with a high processivity reverse transcriptase (RT) to generate a first cDNA strand complementary to the mRNA transcripts;
(c) contacting the first cDNA strand with a high processivity reverse transcriptase (RT) or high processivity DNA polymerase to generate a second cDNA strand complementary to the first cDNA strand;
(d) amplifying the second strand cDNA to form a PCR template and isolating the PCR template.

22. The method of any one of claims 18 to 21, wherein the high processivity RT is Superscript IV, thermostable group II intron RT (TGIRT), or marathon RT.

23. The method of any one of claims 18 to 22, wherein the high processivity DNA polymerase is Klenow exo−, Bst 3.0, or phi29.

24. A method for preparing an mRNA transcriptome library from a tissue sample comprising,
(a) contacting total RNA isolated from the sample with polynucleotide kinase (PNK) to modify 3′ phosphate to a hydroxyl group to generate end repaired total RNA;
(b) contacting the total RNA with polynucleotide kinase (PNK) to modify a 3′ phosphate to a hydroxyl group to generate end repaired total RNA;
(c) contacting the end repaired total RNA with polyadenylate polymerase (PAP) and adenosine nucleotides to generate polyadenylated total RNA;
(d) releasing the polyadenylated total RNA from the tissue sample;
(e) capturing the polyadenylated total RNA on a substrate comprising one or more oligonucleotides comprising a poly T sequence;
(f) depleting ribosomal RNA from the total RNA leaving polyadenylated mRNA;
(g) contacting the polyadenylated mRNA with a reverse transcriptase (RT) to generate a first cDNA strand complementary to the mRNA transcripts;
(h) contacting the first cDNA strand with a high processivity reverse transcriptase (RT) or high processivity DNA polymerase to generate a second cDNA strand complementary to the first cDNA strand to generate PCR templates;
(i) eluting the PCR templates; and
(j) generating an mRNA library from the PCR templates.

25. The method of any one of claims 18 to 24, wherein the substrate is a bead, a bead array, a spotted array, a flow cell, clustered particles arranged on a surface of a chip, a film, or a plate.

26. The method of any one of claims 18 to 25, wherein the sample is a fresh frozen tissue sample or a formalin-fixed paraffin embedded (FFPE) sample.

27. The method of any one of claims 24 to 26, wherein releasing comprises contacting the sample with a lysis buffer, a pemeabilization buffer and/or a reagent to deparaffinize a FFPE sample.

28. The method of any one of claims 24 to 27, wherein when the sample is a FFPE sample on a slide, the method comprises permeabilization and collagenase treatment of the sample on the slide prior to contacting the RNA with PNK.

29. The method of any one of claims 26 to 28, further comprising decrosslinking the FFPE sample, optionally wherein the decrosslinking is carried out using TE buffer, pH 9.

30. The method of any one of claims 24 to 29, wherein the polyA tail is between 3 and 50 nucleotides.

31. The method of any one of claims 24 to 30, wherein generating the RNA library comprises the steps of eluting the polyadenylated total RNA from the substrate and generating the RNA library from the eluted polyadenylated RNA library using a RNA library prep kit.

32. The method of any one of claims 24 to 31, wherein generating the RNA library comprises,
i) contacting the isolated RNA with a reverse transcriptase (RT) to generate a first cDNA strand complementary to the RNA;
ii) contacting the first cDNA strand with a reverse transcriptase (RT) or DNA polymerase to generate a second cDNA strand complementary to the first cDNA strand;
iii) amplifying the second strand cDNA to form a PCR template and isolating the PCR template; and
iv) generating an mRNA library from the PCR templates.

33. The method of any one of claims 24 to 32, wherein the RNA library is an mRNA library.

34. The method of claim 33, wherein the PCR templates are further processed by tagmentation to generate a spatial transcriptomics library.

35. The method of claim 34, wherein the tagmentation comprises on bead tagmentation, wherein the bead comprises a plurality of bead-linked transposomes (BLT).

36. The method of claim 35, wherein the BLT comprises,
i) a plurality of oligonucleotides comprising a first clustering sequence (P7), a first index sequence and a Read 1 sequencing primer (Rd1 SP); and
ii) a plurality of oligonucleotides comprising a second clustering sequence (P5), a second index sequence and a Read 2 sequencing primer (Rd2 SP).

37. The method of any one of claims 24 to 36 wherein reverse transcriptase is a high processivity reverse transcriptase.
